(19) United States
(12) Patent Application Publication
(10) Pub. No.: US 2023/0099102 A1
(43) Pub. Date: Mar. 30, 2023

(54) SOLID CLEANSING COMPOSITION

(71) Applicant: Innospec Active Chemicals LLC, High Point, NC (US)

(72) Inventors: Phillip Loraine Cotrell, Salisbury, NC (US); Derek Michael Overcash, Salisbury, NC (US); Mitchell Jared Cohen, Winston-Salem, NC (US)

(21) Appl. No.: 17/800,038

(22) PCT Filed: Mar. 10, 2021
(86) PCT No.: PCT/GB2021/050600

§ 371 Date: Aug. 16, 2022

Related U.S. Application Data
(60) Provisional application No. 62/988,033, filed on Mar. 11, 2020.

(51) Int. Cl. A61K 8/46; A61K 8/34; A61K 8/73; A61K 8/92; A61Q 19/10; A61Q 5/02

(52) U.S. Cl. CPC A61K 8/463; A61K 8/345; A61K 8/737; A61K 8/922; A61Q 19/10; A61Q 5/02

(57) ABSTRACT

A solid cleansing composition comprising:
(i) from 40 to 60 wt % of at least one acyl isethionate surfactant of the formula (I):

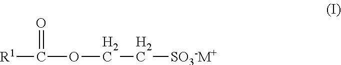

wherein R1 represents an optionally substituted C4-C36 hydrocarbyl group; and
M+ represents a cation;
(ii) from 10 to 25 wt % of a C2-C8 polyhydroxy alcohol; and
(iii) from 10 to 25 wt % of water.

### FIELD OF THE INVENTION

The present invention relates to solid cleansing compositions. The present invention also relates to a method of cleansing skin and/or hair, the use of a solid cleansing composition for cleansing skin and/or hair, a cleansing product comprising a solid cleansing composition, and a method of manufacturing a solid cleansing composition.

### BACKGROUND OF THE INVENTION

Many commonly available cleansing products (such as shampoos, body washes and the like) are in the form of viscous liquid compositions. Such compositions are easy to dispense. However, a user will often pour a much larger volume than they intended of the composition onto their hand prior to application, for example to the hair or body, and thus significant quantities are wasted. Liquid compositions are heavy and often contain large volumes of water. It is expensive and environmentally unfriendly to transport large volumes of liquid, and the use of large quantities of water during manufacturing is detrimental to the environment.

Liquid cleansing compositions are usually packaged in plastic bottles. Plastic bottles are durable, flexible, and easy to manufacture in a variety of shapes. However, most plastic bottles are derived from petrochemicals and not from a sustainable source. Plastic bottles are typically not biodegradable and plastic bottles which are discarded will typically persist in the environment for a long period of time. Plastic bottles may be recycled, but this is energy- and labour-intensive and currently only a small proportion of plastic bottles are recycled.

Solid cleansing compositions offer significant advantages over liquid compositions. They are more compact, require less packaging (especially less plastic packaging) and are easy to transport. In addition, a user typically only uses the amount of the composition needed and thus there is a reduction in waste. Solid cleansing compositions are not stored and used in bottles, which, along with the overall reduction in packaging, makes them easier to use and apply. This provides advantages especially for use by older people and for application to animals. It is easy to provide solid compositions as single use products.

Known solid cleansing compositions include soap and Syndet bars that typically comprise sodium cocoyl isethionate, about 20 wt % soap and about 25 wt % free fatty acid. However, the surfactants in such compositions are quite harsh and may cause irritancy to the skin of a user.

Low-irritancy solid cleansing compositions typically have inadequate foam-forming properties when brought into contact with water and/or the body of a user. It may take a long time and considerable effort for a user to achieve the desired level of foam, and the user may not be able to fully apply the cleansing composition to the desired part of the body.

Therefore, there remains a need for solid cleansing compositions which are low-irritancy, yet quick and easy to use, and which form desirable foams in use.

It is an aim of the present invention to provide an improved solid cleansing composition.

### SUMMARY OF THE INVENTION

According to a first aspect of the present invention, there is provided a solid cleansing composition comprising:

(i) from 40 to 60 wt % of at least one acyl isethionate surfactant of the formula (I):

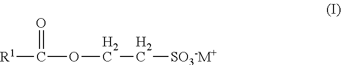

wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water.

According to a second aspect of the present invention, there is provided a method of cleansing skin and/or hair comprising contacting the skin and/or hair with a solid cleansing composition according to the first aspect.

According to a third aspect of the present invention, there is provided a use of a solid cleansing composition according to the first aspect for cleansing skin and/or hair.

According to a fourth aspect of the present invention, there is provided a cleansing product comprising a solid cleansing composition according to the first aspect and packaging.

According to a fifth aspect of the present invention, there is provided a method of manufacturing a solid cleansing composition, the method comprising the steps of:

(a) forming a molten admixture comprising:
(i) from 40 to 60 wt % of at least one acyl isethionate surfactant of the formula (I):

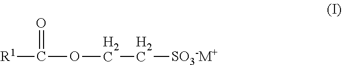

wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water;
(b) transferring the molten admixture into a mould; and
(c) solidifying the molten admixture.

### DETAILED DESCRIPTION OF THE INVENTION

Unless otherwise stated, the following terms used in the specification and claims have the meanings set out below.

The term “fatty acid” is used herein in its ordinary sense, which is well-known to those skilled in the art. Specifically, it refers to a nonesterified fatty acid.

As used herein, the term “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include hydrocarbon groups, i.e. aliphatic (which may be saturated or unsaturated, linear or branched, for example alkyl or alkenyl), alicyclic (for example cycloalkyl, cycloalkenyl) and aromatic (for example phenyl) groups.

As used herein, the term “polyhydroxy alcohol” is used in its ordinary sense, which is well known to those skilled in the art. Specifically it refers to an alcohol having two or more, for example from 2 to 8, such as from 2 to 6, hydroxy groups.

The term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “C4-C36 alkyl” includes C10-C36, C4-C6 alkyl, propyl, isopropyl and t-butyl.

The term “alkenyl” includes both straight and branched chain alkenyl groups. References to individual alkenyl groups such as “propenyl” are specific for the straight chain version only and references to individual branched chain alkenyl groups such as “isopropenyl” are specific for the branched chain version only. For example, “C4-C36 alkenyl” includes C10-C36 alkenyl, C4-C6 alkenyl, propenyl and isopropenyl.

The term “alkoxy” includes both straight and branched chain alkoxy groups. References to individual alkoxy groups such as “propoxy” are specific for the straight chain version only and references to individual branched chain alkyl groups such as “isopropoxy” are specific for the branched chain version only. For example, “C1-C4 alkoxy” includes C1-C2 alkoxy, propoxy, isopropoxy and t-butoxy.

The term “aryl” means a cyclic or polycyclic aromatic ring having from 6 to 12 carbon atoms. Examples of aryl groups include, but are not limited to, phenyl, biphenyl and naphthyl. In a particular embodiment, an aryl group may be phenyl.

The term “C8-C22 alkyl-C6-C12 aryl” means a C6-C12 aryl group covalently attached to a C8-C22 alkyl group, both of which are defined herein.

The term “optionally substituted” with reference to a particular group, such as a hydrocarbyl group, alkyl group, alkenyl group, alkoxy group, or aryl group, means that said group may be substituted or unsubstituted. Suitable substituents may include non-hydrocarbon groups provided that they do not alter the predominantly hydrocarbon nature of the group. Examples of suitable substituents include C1-4 alkoxy, cyano, hydroxy, oxo, halo (especially fluoro and chloro), trifluoromethyl and trifluoromethoxy.

Unless stated to be optionally substituted, the hydrocarbyl groups, alkyl groups, alkenyl groups, alkoxy groups, and aryl groups herein are unsubstituted.

References to a solid composition herein refer to compositions which are in the solid state under normal atmospheric conditions (i.e. at a pressure of 1 atmosphere and 298 K).

References to “soap” herein refer to compounds commonly known as soap, for example the alkali metal, alkaline earth metal, ammonium, ammonium hydroxide and alkanol ammonium salts of aliphatic alkane or alkene monocarboxylic acids.

Throughout this specification, the term “comprising” or “comprises” means including the component(s) specified but not to the exclusion of the presence of other components. The term “consisting essentially of” or “consists essentially of” means including the components specified but excluding other components except for components added for a purpose other than achieving the technical effect of the invention. The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

For the avoidance of doubt, where amounts of components in a composition are described in wt %, this means the weight percentage of the specified component in relation to the whole composition referred to. For example, “wherein the solid cleansing composition comprises from 40 to 50 wt % of at least one acyl isethionate surfactant of the formula (I)” means that 40 to 50 wt % of the solid cleansing composition is provided by at least one acyl isethionate of the formula (I).

In this specification, unless otherwise indicated any amounts referred to relate to the amount of active component present in the composition. The skilled person will appreciate that commercial sources of some of the components referred to herein may include impurities, side-products and/or residual starting material. However, the amounts specified refer only to the active material and do not include any impurity, side-product, starting material or diluent that may be present.

The optional features set out herein may be used either individually or in combination with each other where appropriate and particularly in the combinations as set out in the accompanying claims. The optional features for each exemplary embodiment of the invention, as set out herein are also applicable to any other aspects or exemplary embodiments of the invention, where appropriate. In other words, the skilled person reading this specification should consider the optional features for each aspect or embodiment of the invention as interchangeable and combinable between different aspects of the invention.

### Solid Cleansing Composition

According to a first aspect of the present invention, there is provided a solid cleansing composition. The solid cleansing composition comprises:

(i) from 40 to 60 wt % of at least one acyl isethionate surfactant of the formula (I):

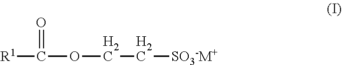

wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water.

The solid cleansing composition of the first aspect may be in any suitable form, for example in the form of a solid bar, block, puck, stick, powder, or leaflet. Suitably the solid cleansing composition is in the form of a bar, block, puck, or stick manufactured in a way known to a person skilled in the art, for example by compacting, pouring and moulding, or extruding. The solid cleansing composition is advantageously lighter and more compact than a liquid cleansing composition comprising the same amount of surfactant. The solid cleansing composition advantageously requires less packaging than liquid cleansing compositions, and may even require no packaging at all.

The solid cleansing composition of the first aspect suitably has a hardness value, as measured according to a modified version of ASTM D1321-10 as described in the Examples, of less than 90 mm, for example less than 80 mm and greater than 0 mm. For example, the solid cleansing composition of the first aspect may have a hardness value in the range of from 0 to about 88 mm, such as from 5 to 80 mm, suitably of from 10 to 70 mm.

Suitably, the solid cleansing composition of the first aspect is for cleansing the skin and/or hair of a human or animal (such as a pet). Suitably, the solid cleansing composition of the first aspect is a personal cleansing composition, for example which is suitable for cleansing skin and/or hair. In some embodiments the solid cleansing composition of the first aspect is a shampoo, a body wash, a hand cleanser, a facial cleanser, a skin cleanser, or a general personal cleanser.

The proportion of the acyl isethionate surfactant of the formula (I), C2-C8 polyhydroxy alcohol, and water in the solid cleansing composition of the first aspect is believed to provide a solid cleansing composition which rapidly forms a stable foam or lather with minimal applied energy and water, said foam or lather being dense and creamy and easily rinsed from hair and skin. The composition provides a highly concentrated surfactant formula and is able to disperse polymer(s) (such as cationic conditioning polymers) in the aqueous phase to provide maximum conditioning to hair and skin upon application.

The solid cleaning composition of the first aspect of the invention comprises from 40 to 60 wt % of at least one acyl isethionate surfactant of the formula (I). Suitably, the solid cleansing composition of the first aspect comprises from 40 to 55 wt % of at least one acyl isethionate surfactant of the formula (I), such as from 40 to 50 wt % of at least one acyl isethionate surfactant of the formula (I), particularly from 43 to 47 wt % of at least one acyl isethionate surfactant of the formula (I).

In the formula (I), the group R1 represents an optionally substituted C4-C36 hydrocarbyl group and M+ represents a cation.

Suitably, R1 represents an optionally substituted C4-C36 alkyl, C4-C36 alkenyl, C6-C12 aryl or C8-C22 alkyl-C6-C12 aryl group. More suitably, R1 represents an optionally substituted C4-C36 alkyl or C4-C36 alkenyl group, especially an optionally substituted C4-C36 alkyl group. Most suitably, R1 represents a C4-C36 alkyl group or C4-C36 alkenyl group, especially a C4-C36 alkyl group.

Suitably, R1 represents an optionally substituted C4-C36 alkyl or C4-C36 alkenyl group, such as an optionally substituted C8-C18 alkyl or C8-C18 alkenyl group.

Suitably, R1 represents a C4-C36 alkyl or C4-C36 alkenyl group, such as a C8-C18 alkyl or C8-C18 alkenyl group.

Suitably, R1 represents an optionally substituted C5-C30 alkyl group, such as an optionally substituted C7-C24 alkyl group, for example an optionally substituted C7-C21 alkyl group, preferably an optionally substituted C7-C17 alkyl group.

Suitably, R1 represents a C5-C30 alkyl group, such as a C7-C24 alkyl group, for example a C7-C21 alkyl group, preferably a C7-C17 alkyl group.

R1 is suitably the residue of a fatty acid. Fatty acids obtained from natural oils often include mixtures of fatty acids. For example, the fatty acid obtained from coconut oil contains a mixture of fatty acids including C12 lauric acid, C14 myristic acid, C16 palmitic acid, C8 caprylic acid, C10 capric and C18 stearic and oleic acid.

R1 may include the residue of one or more naturally occurring fatty acids and/or of one or more synthetic fatty acids. For example, R1 may consist essentially of the residue of a single fatty acid.

Examples of carboxylic acids from which R1 may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Suitably R1 comprises the residue of coco acid, the residue of mixed fatty acids derived from coconut oil or the residue of mixed fatty acids derived from palm kernel oil.

For example, the acyl isethionate surfactant of the formula (I) may be selected from one or more of sodium lauroyl isethionate, sodium cocoyl isethionate and sodium myristoyl isethionate. Sodium cocoyl isethionate is especially preferred.

In some embodiments only a single acyl isethionate surfactant of the formula (I) may be present in the solid cleansing composition of the first aspect. In some embodiments a mixture of two or more acyl isethionate surfactants of the formula (I) may be present. In such embodiments the above amounts refer to the total amounts of all acyl isethionate surfactants of the formula (I) present in the composition.

Suitably, M+ represents a metal cation or an optionally substituted ammonium cation, preferably a metal cation. By “optionally substituted ammonium cation”, we mean to refer to an ammonium cation wherein the nitrogen atom may be substituted with from 1 to 4 optionally substituted hydrocarbyl groups. Suitable ammonium cations include NH4+ and the ammonium cation of triethanolamine. Suitable metal cations include alkali metal cations, for example sodium, lithium and potassium cations, and alkaline earth metal cations, for example calcium and magnesium cations. Preferably, M+ represents a zinc, potassium or sodium cation. Most preferably, M+ represents a sodium cation.

The skilled person will appreciate that when M+ is a divalent metal cation two moles of anion will be present for each mole of cation.

The solid cleansing composition of the first aspect of the invention comprises 10 to 25 wt % of a C2-C8 polyhydroxy alcohol. Suitably, the solid cleansing composition of the first aspect comprises from 10 to 22.5 wt % of a C2-C8 polyhydroxy alcohol, more suitably from 10 to 20 wt % of a C2-C8 polyhydroxy alcohol, such as from 15 to 20 wt % of a C2-C8 polyhydroxy alcohol.

In some embodiments only a single C2-C8 polyhydroxy alcohol may be present in the solid cleansing composition of the first aspect of the invention. In some embodiments a mixture of two or C2-C8 polyhydroxy alcohols may be present. In such embodiments the above amounts refer to the total amounts of all C2-C8 polyhydroxy alcohols present in the composition.

Suitably, the C2-C8 polyhydroxy alcohol comprises a C2-C8 alkane substituted with two or more (such as from 2 to 8, for example from 2 to 6) hydroxy groups.

Preferably, the C2-C8 polyhydroxy alcohol comprises from 2 to 8, such as from 2 to 6 hydroxy groups, for example from 2 to 4 hydroxy groups, such as 2 or 3 hydroxy groups.

The C2-C8 polyhydroxy alcohol suitably comprises a C2-C6 polyhydroxy alcohol, such as a C2-C4 polyhydroxy alcohol, for example a C3-C4 polyhydroxy alcohol.

Examples of suitable C2-C8 polyhydroxy alcohols include glycerine, sorbitol, propylene glycol, butylene glycol, and mixtures thereof. For example, the C2-C8 polyhydroxy alcohol may comprise glycerine.

The solid cleansing composition of the first aspect of the invention comprises from 10 to 25 wt % of water. Suitably, the solid cleansing composition of the first aspect of the invention comprises from 10 to 22.5 wt % of water, more suitably from 10 to 20 wt % of water, such as from 13 to 20 wt % of water, for example from 15 to 20 wt % of water. The solid cleansing composition of the first aspect of the invention may comprise more than 12 wt % of water.

It is believed that the presence of 10 to 25 wt % of water in the solid cleansing composition facilitates the dissolution of the acyl isethionate surfactant of the formula (I) upon contact of the composition with liquid water. The water is also believed to improve the hydration of a conditioning polymer (when present) so as to aid its dispersion in use.

The solid cleansing composition of the first aspect may comprise at least one additional ingredient. By additional ingredient, we mean a component of the composition other than the acyl isethionate surfactant of the formula (I), the C2-C8 polyhydroxy alcohol, and water. Suitably, the composition comprises from 0.001 to 30 wt % of at least one additional ingredient, such as from 5 to 30 wt % of at least one additional ingredient, for example from 10 to 30 wt % of at least one additional ingredient, preferably from 15 to 30 wt % of at least one additional ingredient.

In some embodiments the additional ingredient comprises one or more further surfactants in addition to the acyl isethionate surfactant of the formula (I). Suitably the solid cleansing composition of the first aspect of the invention comprises from 1 to 15 wt % of an additional surfactant, preferably from 2 to 12 wt % of an additional surfactant, such as from 4 to 10 wt % of an additional surfactant.

The additional surfactants may be selected from one or more anionic surfactants, cationic surfactants, non-ionic surfactants and amphoteric surfactants.

Suitable anionic surfactants for use in compositions of the first aspect of the invention include salts of C12-C18 carboxylic acids, ethoxylated carboxylic acids, ester carboxylates and ethoxylated ester carboxylates and sarcosinates. Other suitable anionic surfactants include sulfates and sulfonates, for example alkyl sulfates, alkyl ether sulfates, alcohol sulfates, alcohol ether sulfates, α-olefin sulfonates, linear alkyl sulfonates; and phosphate esters.

Suitable anionic surfactants may be selected from salts of fatty acids; alkali metal salts of mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; alkyl glyceryl ether sulfonates; alpha-olefinsulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, acyl alkyl isethionates, non-acylated alkyl isethionates; fatty acid taurates; acyl taurates; amino acid surfactants such as glutamates and glycinates; products of condensation of fatty acids with oxy- and aminoalkanesulfonic acids; sulfated derivatives of fatty acids and polyglycols; alkyl and acyl sarcosinates; sulfoacetates; alkyl phosphates; alkyl phosphate esters; acyl lactates; alkanolamides of sulfated fatty acids and salts of lipoamino acids. Particularly exemplary salts of the above, where applicable, are the sodium, potassium, ammonium, magnesium and triethanolamine salts. For example, suitable anionic surfactants may comprise acyl alkyl isethionates and acyl taurates. A suitable acyl taurate is sodium methyl cocoyl taurate.

Suitable sulfoacetates include acyl sulfoacetates, particularly sodium acyl sulfoacetates.

Acyl alkyl isethionates for use in compositions of the first aspect of the invention may be of the formula (II):

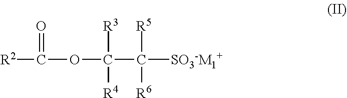

wherein R2 represents an optionally substituted C4-C36 hydrocarbyl group; R3, R4, R5 and R6 each independently represents hydrogen or an optionally substituted C1-C4 alkyl group, provided that at least one of R3, R4, R5 and R6 is not hydrogen; and M1+ represents a cation.

Suitably, R2 is selected from an optionally substituted C4-C36 alkyl, C4-C36 alkenyl, C6-C12 aryl or C8-C22 alkyl-C6-C12 aryl group. More suitably, R2 is selected from an optionally substituted C4-C36 alkyl or C4-C36 alkenyl group. For example, R2 may represent a C4-C36 alkyl or C4-C36 alkenyl group, especially a C4-C36 alkyl group.

Suitably, R2 represents an optionally substituted C5-C30 alkyl group, such as an optionally substituted C7-C24 alkyl group, for example an optionally substituted C7-C21 alkyl group, preferably an optionally substituted C7-C17 alkyl group.

Suitably, R2 represents a C5-C30 alkyl group, such as a C7-C24 alkyl group, for example a C7-C21 alkyl group, preferably a C7-C17 alkyl group.

R2 is suitably the residue of a fatty acid. Fatty acids obtained from natural oils often include mixtures of fatty acids. For example, the fatty acid obtained from coconut oil contains a mixture of fatty acids including C12 lauric acid, C14 myristic acid, C16 palmitic acid, C8 caprylic acid, and C18 stearic and oleic acid.

R2 may include the residue of one or more naturally occurring fatty acids and/or of one or more synthetic fatty acids. For example, R2 may consist essentially of the residue of a single fatty acid.

Examples of carboxylic acids from which R2 may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Suitably R2 comprises the residue of coco acid, the residue of mixed fatty acids derived from coconut oil or the residue of mixed fatty acids derived from palm kernel oil. More suitably, R2 comprises the residue of a saturated fatty acid having 12 carbon atoms.

For example, the acyl alkyl isethionate of the formula (II) may be selected from one or more of sodium lauroyl methyl isethionate, sodium cocoyl methyl isethionate and sodium oleoyl methyl isethionate. Sodium lauroyl methyl isethionate is especially preferred.

When any of R3, R4, R5 and R6 represents an optionally substituted C1-C4 alkyl group, the alkyl group is preferably n-propyl, ethyl or methyl, such as ethyl or methyl, most preferably methyl.

Suitably one of the groups R3, R4, R5 and R6 represents an optionally substituted C1-C4 alkyl group and the remaining groups represent hydrogen. For example, R3 may represent an optionally substituted C1-C4 alkyl group and R4, R5 and R6 may all represent hydrogen.

For example, R5 may represent an optionally substituted C1-C4 alkyl group and R3, R4 and R6 may all represent hydrogen.

Suitably, R3 represents a C1-C4 alkyl group and R4, R5 and R6 all represent hydrogen. Suitably, R5 represents a C1-C4 alkyl group and R3, R4 and R6 all represent hydrogen.

Most suitably, R3 represents a methyl group and R4, R5 and R6 all represent hydrogen. Most suitably, R5 represents a methyl group and R3, R4 and R6 all represent hydrogen.

Suitably, M1+ represents an optionally substituted ammonium cation or, most preferably, a metal cation. Suitable ammonium cations include NH4+ and the ammonium cation of triethanolamine. Suitable metal cations include alkali metal cations, for example sodium, lithium and potassium cations, and alkaline earth metal cations, for example calcium and magnesium cations. More suitably, M1+ represents a zinc, potassium or sodium cation. Most suitably, M1+ represents a sodium cation.

The skilled person will appreciate that when M1+ is a divalent metal cation two moles of anion will be present for each mole of cation.

Acyl alkyl isethionates for use in compositions of the first aspect of the invention preferably comprise sodium acyl methyl isethionates, such as sodium lauroyl methyl isethionate.

The solid cleansing composition of the first aspect of the invention may include a mixture of more than one compound of formula (II). For example, an isomeric mixture of compounds of formula (II) may be present. Such a mixture may include, for example, a compound in which R3 is substituted or unsubstituted C1-C4 alkyl (suitably methyl) and R4, R5 and R6 are all hydrogen and a compound in which R5 is substituted or unsubstituted C1-C4 alkyl (suitably methyl) and R3, R4 and R6 are all hydrogen.

The acyl alkyl isethionate surfactant of formula (II) may comprise the reaction product of sodium methyl isethionate and a fatty acid, that is a compound of formula R2COOCHR3CHR5SO3-M+ in which one of R3 and R5 is methyl and the other is hydrogen. Mixtures of these isomers may be present.

In particular, the solid cleansing composition of the present invention may comprise a mixture of isomers of an acyl alkyl isethionate surfactant of formula (II), that is a compound of formula R2COOCH2CHR5SO3-M+ in which R5 represents a C1-C4 alkyl group (preferably methyl) and a compound of formula R2COOCHR3CH2SO3-M+ in which R3 represents a C1-C4 alkyl group (preferably methyl).

The acyl alkyl isethionate of the formula (II) may be prepared by any of the methods disclosed in the prior art, for example see the methods described in WO 94/09763 and WO 2005/075623.

In some embodiments only a single acyl alkyl isethionate of the formula (II) may be present in the solid cleansing composition of the first aspect of the invention. In some embodiments a mixture of two or more acyl alkyl isethionates of the formula (II) may be present.

Preferred additional anionic detersive surfactants for use in compositions of the first aspect of the invention include alkyl glyceryl ether sulfonate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Suitable non-ionic surfactants for use in compositions of the first aspect of the invention include alcohol alkoxylates such as alcohol ethoxylates, alcohol propoxylates, and ethylene oxide/propylene oxide copolymer derived surfactants, aliphatic esters, aromatic esters, sugar esters, especially sorbitan esters, alkyl polyglucosides, fatty acid alkoxylates such as fatty acid ethoxylates and fatty acid propoxylates or polyethylene glycol esters and partial esters, glycerol esters including glycerol partial esters and glycerol triesters, fatty alcohols (such as cetearyl alcohol, lauryl alcohol, stearyl alcohol, behenyl alcohol), alkanolamides and amineoxides.

Suitable non-ionic surfactants may be selected from the following: reaction products of compounds having a hydrophobic group and a reactive hydrogen atom, for example aliphatic alcohols, acids, amides or alkyl phenols with alkylene oxides, especially ethylene oxide either alone or with propylene oxide (for example alkyl (C6-C22) phenol-ethylene oxide condensates, the condensation products of aliphatic (C8-C18) primary or secondary linear or branched alcohols with ethylene oxide, and products made by condensation of ethylene oxide with the reaction products of propylene oxide and ethylenediamine); long chain tertiary amine oxides, long chain tertiary phosphine oxides and dialkyl sulfoxides; alkyl amine oxides, alkyl amido amine oxides; alkyl tertiary phosphine oxides; alkoxyl alkyl amines; sorbitan; sorbitan esters; sorbitan ester alkoxylates; glycerol ester alkoxylates; sucrose esters; sugar amides, such as a polysaccharide amide; lactobionamides; and alkyl polysaccharide nonionic surfactants, for example alkylpolyglycosides.

Suitable cationic surfactants for use in compositions of the first aspect of the invention are typically based on fatty amine derivates or phosphonium quaternary ions, and quaternary ammonium compounds.

Suitable cationic surfactants for use in compositions of the first aspect of the invention include tertiary amine salts, mono alkyl trimethyl ammonium chloride, mono alkyl trimethyl ammonium methyl sulfate, dialkyl dimethyl ammonium chloride, dialkyl dimethyl ammonium methyl sulfate, trialkyl methyl ammonium chloride and trialkyl methyl ammonium methyl sulfate.

Examples of suitable cationic surfactants include quaternary ammonium compounds, particularly trimethyl quaternary compounds.

Preferred quaternary ammonium compounds include cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen (e.g. bromide), acetate, citrate, lactate, glycolate, phosphate nitrate, sulfate, or alkylsulfate.

Further suitable cationic surfactants include those materials having the CTFA designations Quaternium-5, Quaternium-31 and Quaternium-18. Mixtures of any of the foregoing materials may also be suitable. A particularly useful cationic surfactant for use as a hair conditioning agent is cetyltrimethylammonium chloride, available commercially, for example as GENAMIN CTAC, ex Hoechst Celanese.

Salts of primary, secondary, and tertiary fatty amines are also suitable cationic surfactants. The alkyl groups of such amines preferably have from 12 to 22 carbon atoms, and can be optionally substituted.

Useful cationic surfactants include amido substituted tertiary fatty amines, in particular tertiary amines having one C12 to C22 alkyl or alkenyl chain. Such amines include stearamidopropyIdimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide.

Also useful are dimethylstearamine, dimethylsoyamine, soyamine, myristylamine, tridecylamine, ethylstearylamine, Ntallowpropane diamine, ethoxylated (with 5 moles of ethylene oxide) stearylamine, dihydroxyethylstearylamine, and arachidyl behenylamine.

These amines are typically used in combination with an acid to provide the cationic species. Suitable acids include L-glutamic acid, lactic acid, hydrochloric acid, malic acid, succinic acid, acetic acid, fumaric acid, tartaric acid, citric acid, L-glutamic hydrochloride, and mixtures thereof; more preferably L-glutamic acid, lactic acid, citric acid.

Other useful cationic amine surfactants include those disclosed in U.S. Pat. No. 4,275,055.

Suitable amphoteric surfactants for use in compositions of the first aspect of the invention include those based on fatty nitrogen derivates and those based on betaines.

Suitable amphoteric or zwitterionic surfactants may be selected from betaines, for example alkyl betaines, alkylamidopropyl betaines, for example cocamidopropyl betaine, alkylamidopropyl hydroxy sultaines, alkylamphoacetates, alkylamphodiacetates, alkyl propionates, alkylamphodipropionates, alkylamphopropionates, alkyliminodipropionates and alkyliminodiacetate. Cocamidopropyl betaine is preferred.

Amphoteric or zwitterionic surfactants for use in compositions of the first aspect may include those which have an alkyl or alkenyl group of 7 to 22 carbon atoms and comply with an overall structural formula:

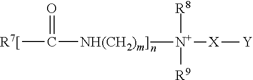

where R7 is alkyl or alkenyl of 7 to 22 carbon atoms; R8 and R9 are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO2 or —SO3.

Amphoteric or zwitterionic surfactants may include simple betaines of formula:

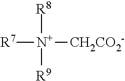

and amido betaines of formula:

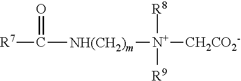

where m is 2 or 3.

In both formulae R7, R8 and R9 are as defined previously. R7 may, in particular, be a mixture of C12 and C14 alkyl groups derived from coconut so that at least half, preferably at least three quarters, of the groups R7 has 10 to 14 carbon atoms. R8 and R9 are preferably methyl.

Amphoteric or zwitterionic surfactants may include sulfobetaines of formula:

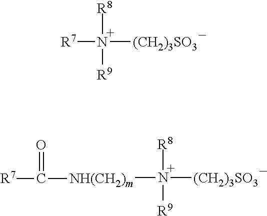

where m is 2 or 3, or variants of these in which
—(CH2)3SO3- is replaced by

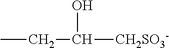

where R7, R8 and R9 in these formulae are as defined previously.

Amphoteric or zwitterionic surfactants may include amphoacetates and diamphoacetates. Amphoacetates generally conform to the following formula:

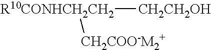

Diamphoacetates generally conform to the following formula:

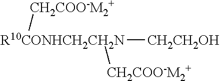

where R10 is an aliphatic group of 8 to 22 carbon atoms and M2+ is a cation such as sodium, potassium, ammonium, or substituted ammonium.

Suitable acetate-based surfactants include lauroamphoacetate; alkyl amphoacetate; sodium alkyl amphoacetate; cocoampho(di)acetate; cocoamphoacetate; disodium cocoamphodiacetate; sodium cocoamphoacetate; disodium cocoamphodiacetate; disodium capryloamphodiacete; disodium lauroamphoacetate; sodium lauroamphoacetate and disodium wheatgermamphodiacetate.

Suitable betaine surfactants include alkylamido betaine; alkyl betaine, C12/14 alkyldimethyl betaine; cocoamidopropylbetaine; tallow bis (hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropl betaine, cocamido betaine, lauryl amido betaine, alkyl amino betaine; alkyl amido betaine; coco betaine; lauryl betaine; diemethicone propyl PG-betaine; oleyl betaine; N-alkyldimethyl betaine; coco biguamide derivative, C8 amido betaine; C12 amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-sodium carboxymethyl-N-carboxymethyl oxyethyl imidazolinium betaine; N-alkyl acid amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; alkamidopropyl hydroxyl sultaine; cocamidopropyl hydroxyl sultaine; undecylenamidopropyl betaine; cocoamidosulfobetaine; alkyl amido betaine; C12/18 alkyl amido propyl dimethyl amine betaine; lauryldimethyl betaine; ricinol amidobetaine; tallow aminobetaine.

Suitable glycinate surfactants include acyl glycinates such as cocoamphocarboxyglycinate; tallowamphocarboxygycinate; capryloamphocarboxyglycinate, oleoamphocarboxyglycinate, bis-2-hydroxyethyl tallow glycinate; lauryl amphoglycinate; tallow polyamphoglycinate; coco amphoglycinate; oleic polyamphoglycinate; N—C10/12 fatty acid amidoethyl-N-(2-hydroxyethyl)-glycinate; N—C12/18-fatty acid amidoethyl-N-(2-hydroxyethyl)-glycinate; dihydroxyethyl tallow gycinate.

Suitable glutamate surfactants include acyl glutamates.

The solid cleansing composition of the first aspect may comprise a chelating agent as an additional ingredient. Suitable chelating agents include ethylenediamine-N,N′-disuccinic acid, methylglycinediacetic acid, glutamic acid N,N-diacetic acid, imino disuccinic acid, diethylene triamine pentaacetic acid, ethylenediamine tetraacetic acid, diethylenetriamine penta methylene phosphonic acid, etidronic acid and anions and mixtures thereof.

Preferred chelants are biodegradable chelants for example ethylenediamine-N,N′-disuccinic acid, methylglycinediacetic acid, glutamic acid N,N-diacetic acid, imino disuccinic acid and anions and mixtures thereof.

Preferably, the solid cleansing composition of the first aspect comprises less than 10 wt % traditional soap compounds. By traditional soap compounds we mean to refer to compounds commonly known as soap, i.e. the alkali metal, alkaline earth metal, ammonium, ammonium hydroxide and alkanol ammonium salts of aliphatic alkane or alkene monocarboxylic acids.

Preferably the compositions of the first aspect comprise less than 5 wt % traditional soap compounds, preferably less than 2.5 wt %, more preferably less than 1 wt % traditional soap compounds. In some embodiments the compositions of the first aspect may be substantially free from traditional soap compounds.

By substantially free from traditional soap compounds we mean that such a product is not deliberately added to the composition. However, the skilled person will appreciate that fatty acids and salts thereof may be present in the composition as side products when providing other surfactants present in the composition, for example the compound of formula (I).

The composition of the first aspect may suitably comprise a conditioning agent as an additional ingredient. Suitable conditioning agents include cationic surfactants, cationic polymers and silicone conditioning agents. Suitable cationic surfactants are as previously defined herein. For example, the additional ingredient may comprise a cationic conditioning polymer

Suitably, the composition of the first aspect comprises from 0.001 to 2 wt % of a conditioning agent (such as a cationic conditioning polymer), such as from 0.001 to 2 wt %, for example from 0.001 to 1 wt %, of a conditioning agent (such as a cationic conditioning polymer), more particularly from 0.1 to 1 wt % of a conditioning agent (such as a cationic conditioning polymer).

For example, by conditioning we mean the conditioning agent may be able to condition hair and/or skin. By conditioning hair we mean to reduce the friction between the hair strands, which improves hair manageability, for example it allows better brushing and/or combing when the hair is wet or dry. By conditioning skin we mean the agent may moisturise the skin, make it more tactile, make it softer or make it more smooth.

Suitable cationic conditioning polymers include cationic polysaccharide polymers, copolymers of 1-vinyl-2-pyrrolidine and 1-vinyl-3-methylimidazolium salt (CTFA name Polyquaternium-16); copolymers of 1-vinyl-2-pyrrolidine and dimethylaminoethyl methacrylate, (CTFA name Polyquaternium-11); cationic diallyl quaternary ammonium-containing polymers in particular (CTFA Polyquaternium 6 and Polyquaternium 7), mineral acid salts of amino-alkyl esters of homo- and copolymers of unsaturated carboxylic acids, for example as described in U.S. Pat. No. 4,009,256; and cationic polyacrylamides, for example as described in WO95/22311.

Cationic polysaccharide polymers suitable for use in compositions of the first aspect include those with an anhydroglucose residual group, such as a starch or cellulose. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e. g. as described in U.S. Pat. No. 3,962,418), and copolymers of etherified cellulose and starch (e. g. as described in U.S. Pat. No. 3,958,581).

A particularly suitable type of cationic polysaccharide polymer that can be used is a cationic guar gum derivative, such as guar hydroxypropyltrimonium chloride or hydroxypropyl guar hydroxypropyltrimonium chloride (commercially available from Solvay in their JAGUAR trademark series). Particularly preferred cationic polymers are JAGUAR C13S, JAGUAR C14, JAGUAR C15, JAGUAR C17 and JAGUAR C16 Jaguar CHT, JAGUAR Excel, JAGUAR C-500 STD and JAGUAR C162.

In some embodiments the solid cleansing composition of the first aspect comprises from 0.001 to 1 wt % of a cationic conditioning polymer, such as from 0.1 to 1 wt % of a cationic conditioning polymer, preferably wherein the cationic conditioning polymer comprises a cationic guar gum derivative.

The solid cleansing compositions of the first aspect may comprise a silicone conditioning agent as an additional ingredient.

Suitable silicone conditioning agents include polydiorganosiloxanes, in particular polydimethylsiloxanes that have the CTFA designation dimethicone. Also suitable for use in compositions of the first aspect (particularly shampoos and conditioners) are polydimethyl siloxanes having hydroxyl end groups, which have the CTFA designation dimethiconol. Also suitable for use in compositions of the first aspect are silicone gums having a slight degree of cross-linking, for example as described in WO 96/31188.

A further preferred class of silicone conditioning agents are amino functional silicones. By “amino functional silicone” is meant a silicone containing at least one primary, secondary or tertiary amine group, or a quaternary ammonium group.

Examples of suitable amino functional silicones include: polysiloxanes having the CTFA designation “amodimethicone”, Specific examples of amino functional silicones suitable for use in compositions of the first aspect are the aminosilicone oils DC2-8220, DC2-8166, DC2-8466, and DC2-8950-114 (all ex Dow Corning), and GE 1149-75, (ex General Electric Silicones).

Suitable quaternary silicone polymers are described in EP530974. A preferred quaternary silicone polymer is K3474, ex Goldschmidt.

Also suitable are emulsions of amino functional silicone oils with non-ionic and/or cationic surfactant.

Pre-formed emulsions of amino functional silicone are also available from suppliers of silicone oils such as Dow Corning and General Electric. Specific examples include DC929 Cationic Emulsion, DC939 Cationic Emulsion, and the non-ionic emulsions DC2-7224, DC2-8467, DC2-8177 and DC2-8154 (all ex Dow Corning).

Also suitable are emulsions of amino functional silicone oils with non-ionic and/or cationic surfactant.

Pre-formed emulsions of amino functional silicone are also available from suppliers of silicone oils such as Dow Corning and General Electric. Specific examples include DC929 Cationic Emulsion, DC939 Cationic Emulsion, and the non-ionic emulsions DC2-7224, DC2-8467, DC2-8177 and DC2-8154 (all ex Dow Corning).

The solid cleansing composition of the first aspect of the invention may comprise a free fatty acid (such as coconut fatty acid) as an additional ingredient. These may be present in an amount of from 0.001 to 15 wt %, preferably from 0.001 to 10 wt %, for example from 0.001 to 7 wt %, such as for example in an amount of from 3 to 7 wt %.

The solid cleansing composition of the present invention may include salts of fatty acids for example salts of monovalent and/or divalent metals. Free fatty acids and salts of fatty acids may be with a side product of the acyl isethionate surfactant of formula (I).

In some embodiments the at least one additional ingredient comprises a sodium acyl methyl isethionate, a sodium alkyl amphoacetate, disodium cocoamphodiacetate, an alkyl betaine, an alkamidopropyl betaine, an alkamidopropyl hydroxyl sultaine, an alkyl propionate, an alkyl sulfate, an alkyl ether sulfate, an alkyl sulfosuccinate, an alkyl ether sulfosuccinate, an acyl taurate, an acyl glycinate, an acyl glutamate, an acyl sarcosinate, an alkyl polyglucoside, an acyl lactylate, a sodium acyl sulfoacetate, an aliphatic ester, an aromatic ester, a glycerol ester, an alcohol alkoxylate, a fatty acid alkoxylate, a fatty acid, or a mixture thereof.

Preferably, the at least one additional ingredient comprises an acyl methyl isethionate, a betaine, an acyl taurate, or a mixture thereof. Suitably, the solid cleansing composition of the first aspect comprises from 4 to 10 wt % of an acyl methyl isethionate, a betaine, an acyl taurate, or a mixture thereof.

The at least one additional ingredient may include further optional ingredients for example fragrances, dyes, hair colourants such as semi-permanent dyes or pigments, hair growth agents, hair growth retardation agents, structuring aids, fillers, slipping agents, plasticising agents, anti-shrinkage agents, binding agents, flowing agents (to aid in processing before compressing into a bar), disintegrants (to aid the dissolution of particularly robust bars), moisturisers, sensory property agents such as cooling agents and warming agents, scalp exfoliant particles, beads or encapsulates which are physically robust in the solid form but rupture on contact with water, opacifying/pearlising agents (e.g. styrene/acrylates copolymer and ethylene glycol distearate), scalp benefit agents, colouring agents, sunscreens, UV filters, preservatives, penetration enhancers (e.g. propylene carbonate, benzyl alcohol etc.), hair styling agents which reside on the hair after rinsing to give the hair stylability and shape longevity, agents for the treatment and/or prevention of head and or pubic lice, agents for the eradication and/or repellence of ticks and other insect pests in human hair and/or animal hair/fur, fungicidal agents, bacteriocidal agents, yeasticidal agents, pH adjustment agents, foam boosting agents (such as cocamide DEA, cocamide MEA, or cocamide MIPA laureth-3), chelating agents, antidandruff agents, active ingredients (such as salicylic acid, benzoyl peroxide, sunscreen, botanical or antimicrobial agents), natural and vegetable oils (including hydrogenated and non-hydrogenated vegetable oils), electrolytes, waxes (such as paraffin, beeswax or Carnauba), polyquats (such as Polyquat 7, Polyquat 10, Polyquat 11 or Polyquat 22), and poloxamers. Components of this type are not limited to those mentioned and will be well known to the person skilled in the art.

The at least one additional ingredient may be selected from one or more of a vegetable oil, an electrolyte, a fragrance, a pigment/colourant, a filler, a wax, a polyquat, a poloxamer, a chelant and an active ingredient.

Suitable fillers include talc, starch, and maltodextrin.

Anti-dandruff agents include piroctone olamine, zinc pyrithione and salicylic acid.

Suitable electrolytes include ionic compounds, for example salts selected from sodium chloride, sodium sulfate, potassium chloride, potassium sulfate, sodium phosphate, disodium phosphate, potassium phosphate, dipotassium phosphate, sodium lactate, and sodium citrate. A preferred electrolyte is sodium chloride.

Suitable natural oils may act as an emollient and include coconut oil, sunflower oil, canola oil, hydrogenate canola oil, palm kernel oil, almond oil, apricot kernel oil, avocado oil and castor oil. Such natural oils may be included in the composition of the first aspect in an amount of 0.001 to 15 wt %.

The solid cleansing composition of the first aspect can be used to clean any suitable substrate, such as the skin or hair, for example the skin or hair of an animal or human. The solid cleansing composition of the first aspect may be provided as a single dose, for example for use in cleaning a desired substrate, such as the skin or hair, for example the skin or hair of an animal or human.

According to a second aspect of the present invention, there is provided a method of cleansing skin and/or hair comprising contacting the skin and/or hair with a solid cleansing composition according to the first aspect.

The method of the second aspect may suitably involve wetting the skin and/or hair and/or wetting the solid cleansing composition with water prior to contacting the cleansing composition with the skin and/or hair.

The method of the second aspect may include the step of rinsing the cleansing composition from the skin and/or hair with water.

Preferably, the method of the second aspect is a method of washing skin and/or hair.

According to a third aspect of the present invention, there is provided a use of a solid cleansing composition according to the first aspect for cleansing skin and/or hair.

According to a fourth aspect of the present invention, there is provided a cleansing product comprising a solid cleansing composition according to the first aspect and packaging. Any suitable packaging may be used and will depend on the exact nature of the product. Suitably, the packaging may be arranged so as to enable a single dose of the cleansing product to be packaged individually

Suitably, the packaging is water-soluble. This allows the product of the fourth aspect to be applied by a user without removing the solid cleansing composition from its packaging. For example, the water-soluble packaging may comprise water-soluble cellulosic packaging.

The product of the second aspect may include instructions for use. These may be provided on the packaging.

According to a fifth aspect of the present invention, there is provided a method of manufacturing a solid cleansing composition, the method comprising the steps of:

(a) forming a molten admixture comprising:
(i) from 40 to 60 wt % of at least one acyl isethionate surfactant of the formula (I):

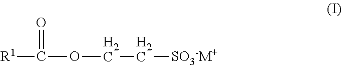

wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water;
(b) transferring the molten admixture into a mould; and
(c) solidifying the molten admixture.

Step (a) of the method of the fifth aspect suitably comprises admixing components which, taken together, comprise:

(i) from 40 to 60 wt % of at least one acyl isethionate surfactant of the formula (I):

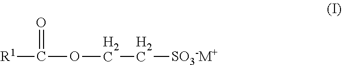

wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water; and
melting the components to form a molten admixture.

The molten admixture may comprise at least one additional ingredient as defined in relation to the first aspect. It is believed that the inclusion of 10 to 25 wt % of water reduces the amount of heat required to form the molten mixture, which improves processability.

Step (a) suitably comprises melting the components by heating the components to a temperature of from 70 to 95° C., such as from 70 to 90° C., for example from 80 to 90° C. This temperature is preferably maintained in step (b).

The mould in step (b) suitably provides the molten admixture with a desired shape, such as a bar, block, puck, or stick.

Step (c) suitably comprises cooling the molten admixture to a temperature at which the molten admixture becomes solid, for example ambient temperature (for example from 20 to 25° C.). Step (c) may comprise allowing the molten mixture to cool naturally over time or actively cooling the molten admixture, for example by refridgeration.

The method of the fifth aspect may further comprise the step of forming the solid obtained in step (c) into a powder, for example by grinding or crushing.

According to a sixth aspect of the present invention, there is provided a method of manufacturing a solid cleansing composition, the method comprising the steps of:

(a) forming an admixture comprising:
(i) from 40 to 60 wt % of at least one acyl isethionate surfactant of the formula (I):

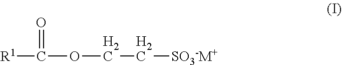

wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water;
(b) heating the admixture; and
(c) extruding the heated admixture.

According to a seventh aspect of the present invention, there is provided a method of manufacturing a solid cleansing composition, the method comprising the steps of:

(a) forming an admixture comprising:
(i) from 40 to 60 wt % of at least one acyl isethionate surfactant of the formula (I):

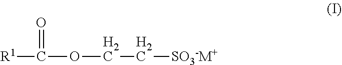

wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water;
(b) compressing the admixture to form the solid cleansing composition.

Preferred features of the second, third, fourth, fifth, sixth and seventh aspects are as defined in relation to the first aspect.

### BRIEF DESCRIPTION OF DRAWINGS

For a better understanding of the invention, and to show how exemplary embodiments of the same may be carried into effect, reference will be made, by way of example only, to the accompanying diagrammatic Figures, in which

FIG. 1 shows the composition prepared as Comparative A in the examples and

FIG. 2 shows the composition prepared as Comparative B in the examples.

### EXAMPLES

The invention will now be described with reference to the following non-limiting examples.

### Examples 1 to 6

Solid cleansing compositions 1 to 6 were prepared comprising the following ingredients, as shown in Table 1:

TABLE 1

| | Example | | | | | |
|---|---|---|---|---|---|---|
| Ingredient | 1 | 2 | 3 | 4 | 5 | 6 |
| Surfactant A | 30% | — | — | — | — | — |
| Surfactant B | — | 30% | | 20% | 16% | — |
| Surfactant C | — | — | 30% | — | — | — |
| Hydrogenated | — | — | — | 10% | 10% | — |
| Canola Oil | | | | | | |
| Water | — | — | — | — | 10% | 20% |
| Glycerin | 18.50% | 18.50% | 18.50% | 18.50% | 10% | 10% |
| ActivSoft | 0.50% | 0.50% | 0.50% | 0.50% | — | — |
| CD | | | | | | |
| Surfactant D | 50% | 50% | 50% | 50% | 54.% | 70% |
| Fragrance | 1.00% | 1.00% | 1.00% | 1.00% | — | |
| Total water | 18 | 18 | 19.5 | 12 | 19.6 | 20 |
| content | | | | | | |

Surfactant A=Sodium lauroyl methyl isethionate

This commercially available surfactant was supplied as an aqueous solution containing 60 wt % water and 34 wt % of the active compound.

Surfactant B=Sodium methyl cocoyl taurate:

This commercially available surfactant was supplied as a paste containing 60 wt % water and 30 wt % of the active surfactant compound.

Surfactant C=Cocamidopropyl betaine

The commercially available surfactant was supplied as an aqueous solution containing 65 wt % water and 30 wt % of active surfactant compound.

Surfactant D=Sodium cocoyl isethionate

This commercially available surfactant was supplied as a solid containing 85 wt % of the active compound.

Activsoft CD is a commercially available Guar Hydroxypropyltrimonium chloride.

### Blending Procedure

### Example 1

Surfactant A was combined with glycerine and mixed until uniform. The admixture was heated to 80-90° C. with smooth agitation. Activsoft CD was slowly blended into system and the admixture was mixed until uniform. Surfactant D was blended in and the admixture was mixed until uniform. Fragrance was blended in and the admixture was mixed until uniform. The temperature was maintained at 80-90° C. The molten admixture was poured into moulds and allowed to cool until solid.

### Example 2

Surfactant B was combined with glycerine and mixed until uniform. The admixture was heated to 80-90° C. with smooth agitation. Activsoft CD was slowly blended into the admixture and the admixture was mixed until uniform. Surfactant D was blended in and the admixture was mixed until uniform. Fragrance was blended in and the admixture was mixed until uniform. The temperature was maintained at 80-90° C. The molten admixture was poured into moulds and allowed to cool until solid.

### Example 3

Surfactant C was combined with glycerine and mixed until uniform. The admixture was heated to 80-90° C. with smooth agitation. Activsoft CD was slowly blended into the admixture and the admixture was mixed until uniform. Surfactant D was blended in and the admixture was mixed until uniform. Fragrance was blended in and the admixture was mixed until uniform. The temperature was maintained at 80-90° C. The molten admixture was poured into moulds and allowed to cool until solid.

### Example 4

Surfactant B was combined with glycerine and mixed until uniform. The admixture was heated to 80-90° C. with smooth agitation. Activsoft CD was slowly blended into the admixture and the admixture was mixed until uniform. Surfactant D was blended in and the admixture was mixed until uniform. Fragrance was blended in and the admixture was mixed until uniform. Hydrogenated canola oil was blended in. The temperature was maintained at 80-90° C. The molten admixture was poured into moulds and allowed to cool until solid.

### Example 5

Surfactant B was combined with water and glycerine and mixed until uniform. The admixture was heated to 80-90° C. with smooth agitation. Surfactant D was blended in and the admixture was mixed until uniform. Hydrogenated canola oil was blended in. The temperature was maintained at 80-90° C. The molten admixture was poured into moulds and allowed to cool until solid.

### Example 6

Glycerine was combined with water and mixed until uniform. The admixture was heated to 80-90° C. with smooth agitation. Surfactant D was blended in and the admixture was mixed until uniform. The temperature was maintained at 80-90° C. The molten admixture was poured into moulds and allowed to cool until solid.

### Example 7

A solid cleansing composition (in Example 7) was prepared, as well as two comparative compositions (A and B), comprising the following ingredients as shown in Table 2:

TABLE 2

| Ingredient | Example 7 | Comparative A | Comparative B |
|---|---|---|---|
| Surfactant D | 51.5 | 40 | 75 |
| Deionized Water | — | 30 | 9 |
| Glycerin | 18.5 | 30 | 9 |
| Surfactant A | 30 | — | 7 |
| Total Water | 18 | 30 | 13.2 |
| Content | | | |

Surfactants A and D are as set out above in relation to Table 1.

### Blending Procedure

### Example 7

Surfactant A was combined with glycerine and mixed until uniform. The admixture was heated to 80-90° C. with smooth agitation. Surfactant D was blended in and the admixture was mixed until uniform. The molten admixture was poured into moulds and allowed to cool until solid.

### Comparative A

Glycerine was combined with water and mixed until uniform. The admixture was heated to 80-90° C. with smooth agitation. Surfactant D was blended in and the admixture was mixed until uniform. The temperature was maintained at 80-90° C. The molten admixture was poured into moulds and allowed to cool until solid.

### Comparative B

Surfactant A was combined with glycerine and mixed until uniform. The admixture was heated to 80-90° C. with smooth agitation. Surfactant D was blended in and the admixture was mixed until uniform. The temperature was maintained at 80-90° C. The molten admixture was poured into moulds and allowed to cool until solid.

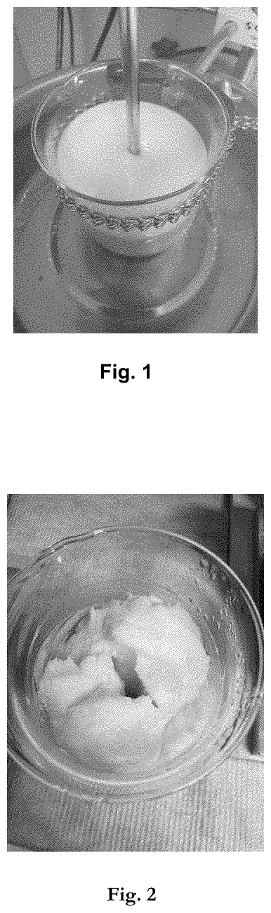

Example 7 provided a solid composition. The composition of Comparative A was very thin when mixed and formed a foam, as shown in FIG. 1. The composition of Comparative A did not set as a hard solid. The composition of Comparative B was an unpourable formulation, which formed clumps and hardened on mixing. In the composition of Comparative B, the sodium cocoyl isethionate did not dissolve. Thus, neither Comparative A nor Comparative B provided a solid cleansing composition.

Hardness testing was conducted on the compositions of Example 7 and Comparative A. The test was conducted using a modified version of ASTM D1321-10, which test uses a penetrometeter to measure how far a needle may penetrate into the bar. The higher the value indicates a softer bar. ASTM D1321-10 is a test method for measuring the hardness of Petroleum Waxes and was modified to be compatible with testing solid cleansing compositions.

The method for testing the hardness of the sample was conducted according to the procedure for testing hardness using a penetrometer (Precision Scientific Instrument Company, Cat. No. 73510) and a standard needle K 17700 (supplied by Koehler Instruments) as described within ASTM D1321-10. The samples tested were 80-90 g of the compositions of Example 7 and Comparative A in a standard bar size. The samples were allowed to equilibrate in the room at 24 (+/−2) ° C. for at least 1 hour prior to testing. A 50 g mass was placed above the needle and plunger assembly making a total load of 100 (+/−0.15) g. The levers were squeezed and held for 5.0 (+/−0.1) seconds while timing this interval with an accurate digital stop watch, and the levers were released after the 5 second interval. The indicator shaft was gently depressed until it stopped and the penetration value was read from the indicator scale. Five tests were made at points equally spaced across the surface of the sample and an average of these readings gave the hardness value. The results are shown in Table 3:

TABLE 3

| Comparative A | Example 7 |
|---|---|
| 97 mm | 53 mm |
| 94 mm | 49 mm |
| 96 mm | 51 mm |
| 102 mm | 52 mm |
| 92 mm | 56 mm |
| Average = | Average = |
| 96.2 mm | 52.2 mm |

Bars with a hardness of around 90 mm and above can be easily squeezed and cracked. Bars with a hardness of about 50 to 60 mm would require much force to break the bar. Thus, Table 3 shows that the composition of Example 7 provides a solid bar of a desirable hardness, whereas the composition of Comparative A does not.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A solid cleansing composition comprising:
(i) from 40 to 60 wt % of at least one acyl isethionate surfactant of the formula (I): 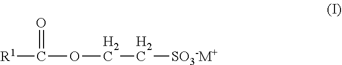
wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water.

2. A composition according to claim 1, comprising from 40 to 50 wt % of at least one acyl isethionate surfactant of the formula (I).

3. A composition according to any preceding claim, wherein R1 represents a C4-C36 alkyl or C4-C36 alkenyl group, preferably a C8-C18 alkyl or C8-C18 alkenyl group, and mixtures thereof.

4. A composition according to any preceding claim, wherein M+ represents a metal cation or an optionally substituted ammonium cation.

5. A composition according to any preceding claim, wherein the at least one acyl isethionate surfactant of the formula (I) is selected from one or more of sodium lauroyl isethionate, sodium cocoyl isethionate, sodium myristoyl isethionate.

6. A composition according to any preceding claim, wherein the C2 to C8 polyhydroxy alcohol comprises one or more of glycerine, sorbitol, propylene glycol and butylene glycol, preferably wherein the C2-C8 polyhydroxy alcohol comprises glycerine.

7. A composition according to any preceding claim, comprising at least one additional ingredient.

8. A composition according to claim 7, wherein the at least one additional ingredient comprises a cationic conditioning polymer, for example comprises from 0.001 to 1 wt % of a cationic conditioning polymer.

9. A composition according to claim 8, wherein the cationic conditioning polymer comprises a cationic guar gum derivative.

10. A composition according to claim 7, wherein the at least one additional ingredient is selected from one or more of a cationic surfactant, a non-ionic surfactant, an anionic surfactant and an amphoteric surfactant.

11. A composition according to claim 7, wherein the at least one additional ingredient is selected from one or more of a vegetable oil, an electrolyte, a fragrance, a pigment/colourant, a filler, a wax, a polyquat, a poloxamer, a chelant and an active ingredient.

12. A composition according to any preceding claim, comprising from 4 to 10 wt % of an acyl methyl isethionate, a betaine, an acyl taurate, or a mixture thereof.

13. A composition according to any preceding claim, comprising free fatty acid in an amount of from 0.001 to 15 wt %.

14. A method of cleansing skin and/or hair comprising contacting the skin and/or hair with a solid cleansing composition according to any preceding claim.

15. Use of a solid cleansing composition according to any of claims 1 to 13 for cleansing skin and/or hair.

16. A cleansing product comprising a solid cleansing composition according to any of claims 1 to 13 and packaging.

17. A cleansing product according to claim 16, wherein the packaging is water-soluble.

18. A method of manufacturing a solid cleansing composition, the method comprising the steps of:
(a) forming a molten admixture comprising:
(i) from 40 to 60 wt % of at least one acyl isethionate surfactant of the formula (I): 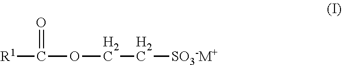
wherein R1 represents an optionally substituted C4-C36 hydrocarbyl group; and M+ represents a cation;
(ii) from 10 to 25 wt % of a C2-C8 polyhydroxy alcohol; and
(iii) from 10 to 25 wt % of water;
(b) transferring the molten admixture into a mould; and
(c) solidifying the molten admixture.
